(19) United States
(12) Patent Application Publication
(10) Pub. No.: US 2006/0016708 A1
(43) Pub. Date: Jan. 26, 2006

(54) Transparent autoclavable bag

(75) Inventor: Brian Scott Ingraham, Madison, WI (US)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Amcor Flexibles Healthcare, Inc.

(21) Appl. No.: 10/896,090

(22) Filed: Jul. 21, 2004

(51) Int. Cl. A61B 19/02; B65D 73/00

(52) U.S. Cl. 206/439; 206/484.1

(57) ABSTRACT

An autoclavable sterilization bag is provided having first and second composite film laminates positioned in opposing relation and sealed to one another to form a pouch. At least the first laminate comprises a heat sealable transparent thermoplastic polymer inner layer forming an inner surface of the laminate and a transparent polymer film outer layer. A transparent barrier layer is located between the inner and outer layers and formed of molecularly oriented polychlorotrifluoroethylene (PCTFE). In advantageous specific embodiments of the invention, the heat sealable transparent thermoplastic polymer inner layer is a polyolefin film, and the transparent polymer outer layer is a film selected from the group consisting of polyethylene terephthalate, nylon, polypropylene, polyethylene and cellophane.

### FIELD OF THE INVENTION

The invention relates to a sterilization bag for medical applications, and to a composite film laminate suitable for producing such a bag.

### BACKGROUND OF THE INVENTION

Many healthcare products, including medical devices and pharmaceutical solutions, are stored until ready for use inside of sealed sterilization bags. Typically, the bags are in the form of a pouch, open along one side for receiving the medical instrument or other medical supply. The bag is then sealed and subjected to sterilization by exposure to gamma irradiation, electron beam, ultraviolet radiation, ethylene oxide, autoclaving, or other sterilization procedures.

In order to withstand the severe conditions of sterilization while providing high moisture barrier properties and long shelf life, sterilization bags this type have traditionally used composite laminate films with a barrier layer of either a metal foil or a metallized film. A disadvantage this type of laminate structure is that the bag is not transparent and therefore does not reveal the contents of the bag. To address this problem, some sterilization bags have provided a small window formed of a transparent barrier material. However, in order to maintain high barrier properties for the bag, the transparent window is kept relatively small in size. It would be desirable for a sterilization bag to have at least one transparent side to more clearly reveal the contents of the bag.

### SUMMARY OF THE INVENTION

The present invention provides a transparent high barrier laminate material that can be suitably fabricated into sterilization bags, providing the ability to see the contents of the bag. The high barrier properties are achieved by using as the barrier layer a molecularly oriented polychlorotrifluoroethylene (PCTFE) film layer.

The autoclavable sterilization bag of the present invention comprises first and second composite film laminates positioned in opposing relation and sealed to one another to form a pouch. At least the first laminate comprises a heat sealable transparent thermoplastic polymer inner layer forming an inner surface of the laminate and a transparent polymer film outer layer. A transparent barrier layer is located between the inner and outer layers and formed of molecularly oriented polychlorotrifluoroethylene (PCTFE). In advantageous specific embodiments of the invention, the heat sealable transparent thermoplastic polymer inner layer is a polyolefin film, polyolefin copolymer, or coextrusions of either, and the transparent polymer outer layer is a film selected from the group consisting of polyethylene terephthalate, nylon, polypropylene, polyethylene and cellophane.

Polychlorotrifluoroethylene (PCTFE) fluoropolymer films are manufactured and sold by Honeywell Inc. under the trademark Aclar®. Non-oriented PCTFE films are used extensively by pharmaceutical companies in manufacturing transparent vacuum-formed blister packages for pharmaceuticals and for other healthcare packaging. However, PCTFE films have seen limited success in non-forming barrier applications in the flexible packaging industry.

The present invention is based upon the discovery that molecularly oriented PCTFE films provide a clear structure with sufficient barrier properties to replace metal foil in even the most demanding barrier applications utilizing ethylene oxide, gamma sterilization, e-beam sterilization, and autoclave sterilization techniques, as well as in non-sterilized applications. Molecularly orienting the PCTFE film decreases the moisture permeation rate significantly as compared to standard non-oriented PCTFE film and provides a very durable barrier layer with superior flex crack resistance.

For most polymer films, the process of molecularly orienting the film results in the film having poor dimensional stability at elevated temperature. When the film is reheated, the molecular chains tend to try to revert to their original non-oriented state, resulting in a dimensional change in the film. When the film is a component of a composite laminate, the dimensional change can result in warping, curling or other unsightly and undesirable changes in the product. Molecularly oriented PCTFE film has surprisingly good thermal dimensional stability, and thus is well suited for use in applications involving exposure to high temperatures, such as in an autoclave sterilization process.

The present invention also provides a dimensionally stable autoclavable composite film laminate that can be used in the fabrication of sterilization bags or other articles. The laminate comprises a heat sealable transparent thermoplastic polymer inner layer forming an inner surface of the laminate, a transparent polymer film outer layer, and a transparent barrier layer between the inner and outer layers and formed of molecularly oriented polychlorotrifluoroethylene (PCTFE). In advantageous embodiments of the invention, the heat sealable transparent thermoplastic polymer inner layer is a film or coating of a polyolefin, polyolefin copolymers, or coextrusions of either, and the transparent polymer outer layer is a film selected from the group consisting of polyethylene terephthalate, nylon, polypropylene, polyethylene and cellophane. The laminate may suitably include a layer of adhesive adhering the polyolefin film inner layer to the transparent barrier layer. The laminate may further include a layer of adhesive adhering the transparent polymer outer film layer to the barrier layer. In one specific embodiment, the dimensionally stable autoclavable composite film laminate comprises a heat sealable transparent polypropylene inner layer forming an inner surface of the laminate, a transparent biaxially oriented polyethylene terephthalate film outer layer, and a transparent barrier layer between the inner and outer layers and formed of molecularly oriented polychlorotrifluoroethylene (PCTFE).

### BRIEF DESCRIPTION OF THE DRAWINGS

Having thus described the invention in general terms, reference will now be made to the accompanying drawings, which are not necessarily drawn to scale, and wherein:

FIG. 1 is a top plan view of a sterilization bag in accordance with one embodiment of the present invention.

FIG. 2 is a cross-sectional view of a heat sealed edge portion of the bag taken substantially along the line 2-2 of FIG. 1.

FIG. 3 is a cross-sectional view showing one embodiment of a dimensionally stable composite film laminate useful in producing the sterilization bag of FIG. 1.

FIG. 4 is a cross-sectional view showing a second embodiment of a dimensionally stable composite film laminate in accordance with the present invention.

### DETAILED DESCRIPTION OF ILLUSTRATIVE EMBODIMENTS

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

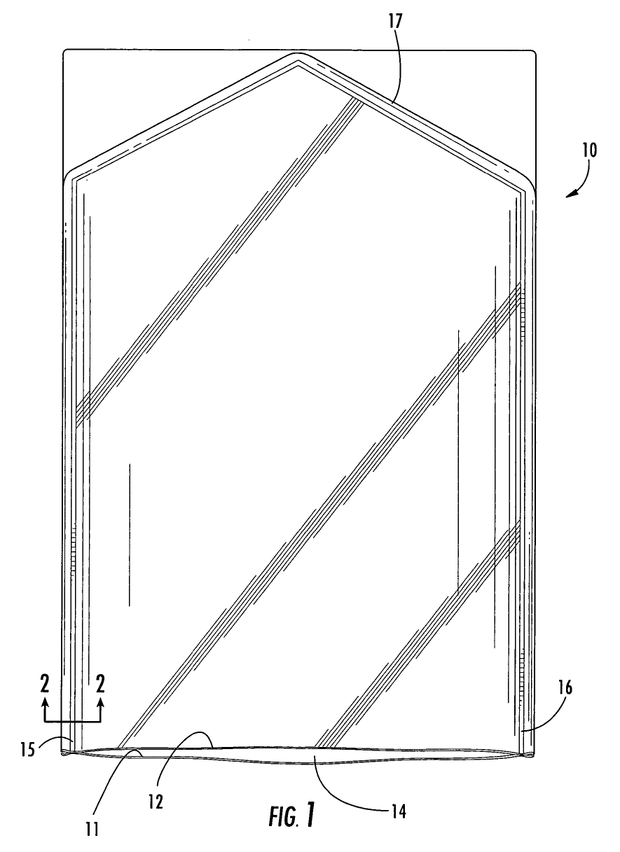

In FIG. 1, the reference character 10 indicates an autoclavable sterilization bag in accordance with the present invention. The bag 10 is formed from first and second composite film laminates 11, 12 positioned in opposing face-to-face relation and sealed to one another along three sides to form a pouch-like structure, leaving an opening 14 along the remaining side suitable for receiving medical instruments or other articles for being sterilized. In the embodiment shown, the film laminates 11, 12 are generally rectangular in shape and they are sealed to one another along seal areas 15,16 adjacent opposite side edges of the bag, and along an angularly extending seal area 17 adjacent the top edge of the bag. After the bag 10 has been filled with its contents by the user, it may be sealed to close the opening 14. Sealing of the film laminates 11, 12 to one another may be carried out using conventional heat or ultrasonic sealing equipment as is well known in the art.

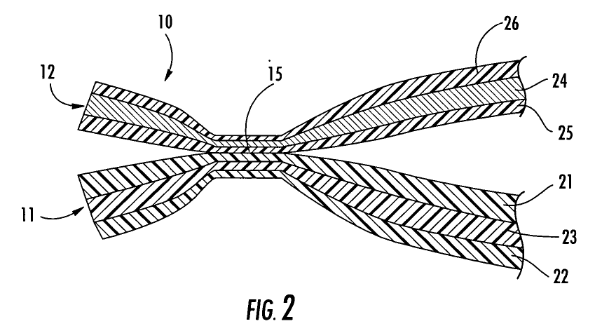

As shown in greater detail in FIG. 2, the film laminate 11 includes three film layers, a heat sealable transparent thermoplastic polymer film inner layer 21 that forms an inner surface of the laminate, a transparent polymer film outer layer 22, and a transparent barrier film layer 23 between the inner and outer film layers 21, 22. The three film layers 21, 22, 23 are laminated to one another by adhesive. The thermoplastic polymer film outer layer 21 has an exposed surface that will soften and become adhesive upon exposure to heat or ultrasonic energy. The film layer 21 is preferably a polyolefin material, suitable examples of which include polypropylene, polyethylene, ethylene copolymers such as EAA, EMA, EVA and ionomer resins such as Surlyng from DuPont or Iotek™ from ExxonMobil. A particularly suitable film material for the inner film layer 21 is a cast polypropylene film made using a cast film extrusion process. The film layer 21 may suitably have a thickness of from about 0.5 mils to about 4.0 mils, more preferably about 1.5 to about 3.0 mils, and most preferably about 2 mils.

The transparent outer film layer 22 imparts strength, puncture resistance, dimensional stability and durability to the film laminate. The film layer 22 also assists in giving the laminate resistance to shrinkage when heated to elevated temperature. Suitable materials for the outer film 22 include polyethylene terephthalate (PET), nylon, polypropylene, polyethylene and cellophane. Particularly preferred are biaxially oriented films such as biaxially oriented PET and biaxially oriented nylon. The outer film layer 22 may have a thickness of from about 0.36 to 2.0 mils, more preferably from about 0.48 to 1.0 mils, and most preferably about 0.48 mils (48 ga).

The transparent barrier layer 23 imparts moisture barrier properties to the laminate. Barrier layer 23 is a molecularly oriented polychlorotrifluoroethylene (PCTFE) fluoropolymer film. The PCTFE film is transparent, biochemically inert, chemical resistant and free from plasticizers and stabilizers. Preferably the molecularly oriented PCTFE film is a monoaxially oriented film. The film may suitably have a thickness of from about 0.60 to 3 mils, more preferably from about 1 to 2 mils, and most preferably a thickness of about 1.5 mils. PCTFE fluoropolymer films are sold by Honeywell, Inc. under the Aclar® trademark.

The sealable inner film layer 21 may be laminated to the barrier layer 23 with an adhesive, using conventional lamination techniques. The adhesive may be applied using known processes such as spraying, roll coating, knife over roll coating, wire rod coating, or gravure coating. Suitable adhesives include solvent based, water based or solventless adhesives including acrylic adhesives, epoxy cured polyester urethanes, moisture cured polyester urethanes and isocyanate terminated polyester adhesives. Alternatively, the inner layer 21 can be formed directly on the barrier layer 23 by extrusion coating. The transparent outer layer 22 can be laminated directly to the barrier layer 23 using known adhesives and techniques as described above. If desired, the inner surface of the transparent outer layer 22 may be reverse printed prior to laminating to provide a layer of printing with graphics or other information. The outer layer may also be surface printed prior to or post lamination.

The film laminate 12 in the embodiment shown in FIG. 2 includes a metal foil layer 24 with a sealable inner surface defined by a sealable film layer 25 of a thermoplastic polymer, such as a polyolefin. Alternatively, the sealable layer 25 can be a coating of a thermoplastic polymer such as a polyolefin. The foil layer 24 may comprise an aluminum foil having a thickness of from 0.275 mil to 1.50 mil. The sealable layer 25 may suitably have a thickness of about 0.5 mil to 4.0 mil. The foil layer 24 provides moisture barrier properties to the laminate. The coating layer 25 facilitates obtaining a strong seal with the heat sealable inner layer 21 of composite laminate 11. The laminate 12 additionally includes a protective outer film layer 26 formed of biaxially oriented PET laminated by an adhesive to the surface of the foil layer 24. The outer film layer 26 may be optionally reverse or surface printed with graphics or other information. In alternative embodiments, the metal layer may comprise a vacuum metallized moisture barrier layer deposited on a film layer such as PET.

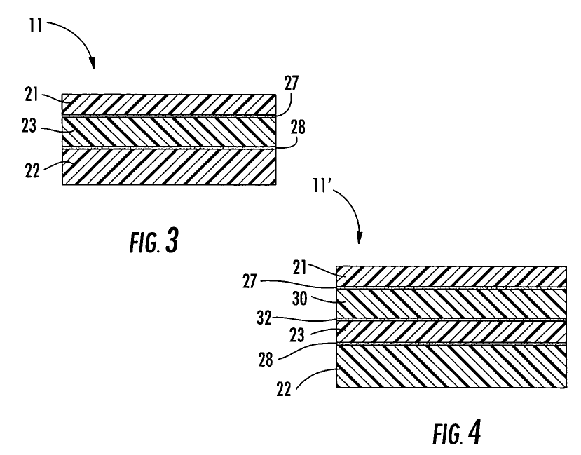

FIG. 3 shows a cross-section of the composite film laminate 11 in greater detail. It will be seen that the inner layer 21 is laminated to the intermediate barrier layer 23 by an adhesive layer 27. The outer layer 22 is laminated to the opposite surface of the intermediate barrier layer 23 by an adhesive layer 20.

FIG. 4 shows an alternative construction for a transparent composite film laminate 11′ which is similar in many respects to the laminate 11 shown in FIGS. 2 and 3. To avoid repetitive description, corresponding reference numbers are used to identify corresponding elements wherever applicable. This embodiment differs over that of FIG. 3 in that there is an additional intermediate film layer 30 located between the heat sealable inner layer 21 and the barrier layer 23. This intermediate barrier layer may suitably comprise a biaxially oriented nylon film or a biaxially oriented PET film, or biaxially oriented polypropylene. It may suitably have a thickness of from 0.36 to 2.0 mils. It is laminated to the inner film layer 21 by an adhesive layer 27 and to the intermediate barrier layer 23 by an adhesive layer 32.

In the embodiment shown and described, the autoclavable bag 10 has a transparent composite film laminate 11 on one side and an opaque metal layer-containing laminate 12 on the opposite side. The article contained within the bag is readily visible through the transparent film laminate 11. However, bags in accordance with the present invention can also be produced using the transparent composites film laminate 11 for both the front and back side of the bag.

### EXAMPLES

### Example 1

A dimensionally stable composite film laminate was manufactured by first laminating a 0.48 mil biaxially oriented transparent polyethylene terephthalate (PET) film to a 1.5 mil thick monoaxially oriented transparent Aclar® polychlorotrifluoroethylene (PCTFE) film to form a two-layer composite. Rolls of the PET film and the Aclarg film were each mounted on unroll stands. The PET film was unrolled and directed across a gravure coating apparatus and a 1.5 lb/ream layer of urethane adhesive was applied to one surface. The Aclar® film was unrolled and then brought into contact with the adhesive-coated surface of the PET film, and the films were directed through a nip formed between two smooth surface rolls. A 2.0 mil polypropylene transparent film made using a cast film extrusion process was then laminated to the exposed surface of the Aclar® film by a similar laminating procedure. The resulting composite laminate exhibited high moisture barrier properties and excellent dimensional stability at elevated temperature. The 1.5 mil machine direction oriented Aclar® layer provided a moisture barrier of 0.0077 g/100 in²/24 hours at 100° F. and 100% relative humidity.

### Example 2

A laminating procedure similar to that described in FIG. 1 was used to produce the following metal laminate: 48 gauge biaxially oriented transparent PET film/adhesive/35 gauge aluminum foil/adhesive/2.0 mil cast polypropylene This tri-laminate was positioned opposite the laminate of Example 1, with the polypropylene surfaces facing one another and the two films were contacted with a heat sealing die to seal the two films together into a pouch of the configuration generally similar to that shown in FIG. 1.

### Example 3

A metal laminate of the following structure can be produced by a procedure similar to example 2: 0.48 mil biaxially oriented transparent PET film with a coating of aluminum deposited on one surface by vacuum metallization to an optical density of 2.8/adhesive/2.0 mil cast polypropylene. A pouch is fabricated from this laminate and the laminate of Example 1.

### Example 4

A pouch is produced by the procedure generally described Example 2, except that the following transparent laminate structure is substituted for the transparent laminate of example 1: 48 gauge biaxially oriented PET/adhesive/1.5 mil oriented Aclar® PCTFE film/adhesive/0.6 mil biaxially oriented nylon film/adhesive/2.0 mil cast polypropylene.

### Example 5

A pouch is produced by the procedure generally described example 2, except that the following transparent laminate structure is substituted for the transparent laminate of Example 1: 48 gauge biaxially oriented PET/adhesive/1.5 mil oriented Aclar® PCTFE film/adhesive/0.48 mil biaxially oriented PET film/adhesive/2.0 mil cast polypropylene.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. An autoclavable sterilization bag comprising first and second composite film laminates positioned in opposing relation and sealed to one another to form a pouch, and wherein at least said first laminate comprises a heat sealable transparent thermoplastic polymer inner layer forming an inner surface of the laminate, a transparent polymer film outer layer, and a transparent barrier layer between said inner and outer layers and formed of molecularly oriented polychlorotrifluoroethylene (PCTFE).

2. A bag according to claim 1, wherein said heat sealable transparent thermoplastic polymer inner layer is a polyolefin film, and said transparent polymer outer layer is a film selected from the group consisting of polyethylene terephthalate, nylon, polypropylene, polyethylene and cellophane.

3. A bag according to claim 2, including a layer of adhesive adhering said polyolefin film inner layer to said transparent barrier layer.

4. A bag according to claim 3, including a layer of adhesive adhering said transparent polymer outer film layer to said barrier layer.

5. A bag according to claim 2, including an intermediate polymer film layer between said polyolefin film inner layer and said barrier layer, a layer of adhesive between said polyolefin film inner layer and said intermediate polymer film layer, and a layer of adhesive between said intermediate film layer and said barrier layer.

6. A bag according to claim 5, wherein said intermediate polymer film layer is a biaxially oriented nylon film.

7. A bag according to claim 2, including a print layer reverse printed onto the interior surface of said transparent outer film layer.

8. A bag according to claim 1, wherein said heat sealable transparent thermoplastic polymer inner layer is a cast polypropylene film bonded directly to one surface of said barrier layer by an adhesive, and said transparent polymer outer layer is a biaxially oriented polyethylene terephthalate film bonded directly to an opposite surface of said barrier layer by an adhesive.

9. A bag according to claim 1, wherein said second composite film laminate includes a metal layer.

10. A bag according to claim 9, wherein said second composite film laminate includes a heat sealable polymer film inner layer adhered to one surface of said metal layer, and a biaxially oriented polymer film outer layer adhered to an opposite surface of said metal layer.

11. A bag according to claim 1, wherein said second composite film laminate comprises a heat sealable transparent thermoplastic polymer inner layer forming an inner surface of the laminate, a transparent polymer film outer layer, and a transparent barrier layer between said inner and outer layers and formed of molecularly oriented polychlorotrifluoroethylene (PCTFE).

12. An autoclavable sterilization bag comprising first and second composite film laminates positioned in opposing relation and sealed to one another along peripheral edge portions to form a pouch, and wherein at least said first laminate comprises a heat sealable transparent polypropylene inner layer forming an inner surface of the laminate, a transparent biaxially oriented polyethylene terephthalate film outer layer, and a transparent barrier layer between said inner and outer layers and formed of molecularly oriented polychlorotrifluoroethylene (PCTFE).

13. A bag according to claim 12, including a first adhesive layer located between and adhering together said polypropylene inner layer and said transparent barrier layer, and a second adhesive layer located between and adhering together said polyethylene terephthalate film outer layer and said transparent barrier layer.

14. A dimensionally stable autoclavable composite film laminate comprising a heat sealable transparent thermoplastic polymer inner layer forming an inner surface of the laminate, a transparent polymer film outer layer, and a transparent barrier layer between said inner and outer layers and formed of molecularly oriented polychlorotrifluoroethylene (PCTFE).

15. A laminate according to claim 14, wherein said heat sealable transparent thermoplastic polymer inner layer is a polyolefin film, and said transparent polymer outer layer is a film selected from the group consisting of polyethylene terephthalate, nylon, polypropylene, polyethylene and cellophane.

16. A laminate according to claim 15, including a layer of adhesive adhering said polyolefin film inner layer to said transparent barrier layer.

17. A laminate according to claim 16, including a layer of adhesive adhering said transparent polymer outer film layer to said barrier layer.

18. A dimensionally stable autoclavable composite film laminate comprising a heat sealable transparent polypropylene inner layer forming an inner surface of the laminate, a transparent biaxially oriented polyethylene terephthalate film outer layer, and a transparent barrier layer between said inner and outer layers and formed of molecularly oriented polychlorotrifluoroethylene (PCTFE).

19. A laminate according to claim 18, including a layer of adhesive adhering said polypropylene inner layer to one surface of said barrier layer, and a layer of adhesive adhering said polyethylene terephthalate film outer layer to the opposite surface of said barrier layer.

20. A laminate according to claim 18, including an intermediate polymer film layer between said polypropylene film inner layer and said barrier layer, a layer of adhesive between said polypropylene film inner layer and said intermediate polymer film layer, and a layer of adhesive between said intermediate film layer and said barrier layer.
